(19) United States
(12) Patent Application Publication
(10) Pub. No.: US 2026/0034312 A1
(43) Pub. Date: Feb. 5, 2026

(54) NEEDLE PROTECTOR SYSTEM

(71) Applicant: Kurin, Inc., San Diego, CA (US)

(72) Inventors: Nolan Pupping, Carlsbad, CA (US); Kevin Nason, Phoenix, AZ (US); Jeffrey Veloz, San Diego, CA (US)

(73) Assignee: Kurin, Inc., San Diego, CA (US)

(21) Appl. No.: 19/248,192

(22) Filed: Jun. 24, 2025

Related U.S. Application Data
(60) Provisional application No. 63/678,761, filed on Aug. 2, 2024.

(51) Int. Cl. A61M 5/32

(52) U.S. Cl. CPC A61M 5/3202; A61M 5/3245

(57) ABSTRACT

Embodiments of needle protectors are disclosed for use with a needle assembly that includes a needle, a housing from which the needle extends, and an actuator for activating a safety mechanism following use of the needle. A needle protector can have a sleeve configured with a lumen sized to accept the needle and a length that extends at least partially over the needle. A cuff can be connected to the sleeve and having a cavity shaped to receive a portion of the housing. An actuator cover can extend from the cuff, opposite the sleeve, and configured to cover the actuator when the needle protector is in place over the needle.

### RELATED APPLICATION(S)

This application claims priority to and the benefit of U.S. Provisional Application No. 63/678,761, filed Aug. 2, 2024, titled “Needle Protector System,” which is hereby incorporated by reference.

### DESCRIPTION OF THE RELATED ART

Needles and needle assemblies can be utilized in various venipuncture applications such as blood draws, intravenous delivery of medicine or fluids, etc. When drawing blood, a physician can expose a needle to be inserted into the patient, insert the needle, perform the blood draw, and remove the needle. The needle can then be disposed of at the appropriate time in a safe and sanitary manner.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram depicting a simplified perspective view of a needle assembly in accordance with certain aspects of the present disclosure,

FIGS. 2A and 2B are simplified cross-sectional views of a needle assembly in a packaged mode and in a safety mode in accordance with certain aspects of the present disclosure,

FIG. 3 is a diagram depicting a simplified perspective and side view of a needle protector in accordance with certain aspects of the present disclosure,

FIG. 4 is a diagram depicting a simplified perspective and side view of a needle protector over a needle shroud in accordance with certain aspects of the present disclosure, and

FIG. 5 is a diagram depicting a simplified side sectional view of a needle protector configured to reset an extended needle shield in accordance with certain aspects of the present disclosure.

### DETAILED DESCRIPTION

The present disclosure describes embodiments of devices that can be utilized with a needle assembly to facilitate safe and proper use of needle assemblies that include actuation mechanisms such as for needle retraction or safety shield deployment. One example of such a device can be a needle protector that protects a needle (to avoid an unintentional needle puncture) and/or covers an actuator (to prevent inadvertent actuation by a user).

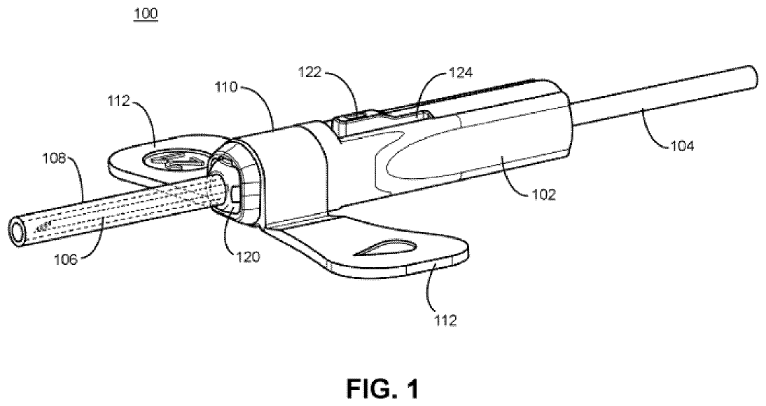

FIG. 1 is a diagram depicting a simplified perspective view of a needle assembly. Needle assembly 100 can include a housing 102 that houses a hub to which tubing 104 or a cannula can be connected. Tubing 104 can in turn be connected at a proximal end to a fluid collection device, a blood sample optimization device, or any other device or receptacle. The tubing can then be used to convey fluids such as blood from needle assembly 100.

The hub within housing 102 can also support needle 106. In some embodiments, when needle assembly 100 is packaged and delivered, needle 106 can be covered by a needle shroud 108. Needle shroud 108 can be, for example, a piece of semi-rigid tubing that can be removed by a technician, but which then protects the technician and others from an accidental needle stick before use.

Needle assembly 100 can also include a wing assembly having collar 110 that at least partially circumscribes the housing 102 and one or more wings 112 extending laterally from collar 110 and housing 102. Collar 110 can be seated in a groove, channel, or cut-in formed on housing 102. As shown in the example of FIG. 1, collar 110 is located at a distal end of housing 102. While FIG. 1 depicts an embodiment that includes collar 110, other embodiments may forego collar 110 and attach the wings 112 to the housing 102 directly such as with adhesive or other methods.

Wings 112 can be grasped by a technician to manipulate and guide the needle assembly 100, and specifically the needle 106. Wings 112 and collar 110 can be formed of an elastomeric material, such as a rubber or plastic, which provides wings 112 in a laterally-extended position unless grasped and manipulated by the technician to a different orientation or position. In their default orientation, wings 112 can also provide a base by which needle assembly 100, and thereby needle 106, can be secured on or in a patient, respectively. For example, one or more of wings 112 can be taped to a patient's skin proximate the venipuncture of needle 106, to secure needle 106 in the venipuncture.

Needle assembly 100 can include a needle shield 120 that can be originally retracted and locked within housing 102, for example by a lock-in latch. In the example of FIG. 1, upon depression of an actuator (such as button 122), the lock-in latch is released allowing a spring to extend needle shield 120 over needle 106. Button 122 can be formed from a cut-out 124 of housing 102 and can include a step or other heightened region for being conveniently depressed by a technician. Cut-out 124 can thereby surround button 122 on one or more sides. Also, the button 122 can be configured (e.g., by being a function of its length and thickness) to require a predetermined force to be depressed. Once deployed and extended, the needle shield 120 can be locked into place by a lock-out latch that resists retraction of needle shield 120.

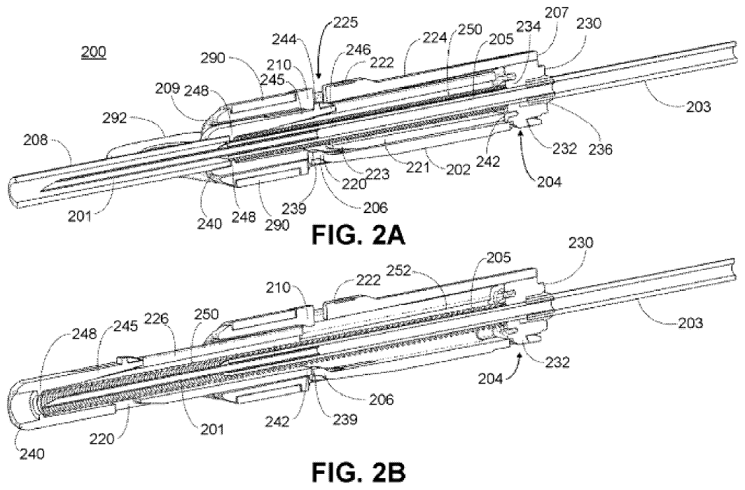

FIGS. 2A and 2B are simplified cross-sectional views of a needle assembly in a packaged mode and in a safety mode. As shown in FIGS. 2A-2B, needle assembly 200 can include housing 202, hub 230 fixed to and housed by housing 202 and which connects outer tubing 203 (or outer cannula) with inner tubing 205 (or inner cannula), and a needle 201 connected in a fixed position relative to hub 230 and housing 202 via the inner tubing 205. In some implementations, needle 201 can be connected directly to the hub 230. Housing 202, hub 230, and needle shield 240 can be formed of a rigid or semi-rigid material, such as a plastic (e.g., polycarbonate).

Housing 202 can include a seat (e.g., a groove or indentation) on an outer surface to accommodate a collar 290 that is attached to oppositely-extending wings (e.g., as depicted in FIG. 1). A technician can hold the collar to move and manipulate the needle assembly 200. The needle assembly can be secured to a patient, such as by the wings being taped to the patient's skin proximate a venipuncture, for example.

The needle assembly 200 can further include a needle shield 240 that is initially retracted and locked, against the force of a compressed spring 250, at least partially within housing 202 so as to expose needle 201 that extends from distal end 209 of housing 202. In the packaged mode, and before use and venipuncture of a patient, needle 201 can be covered with shroud 208 that can extend substantially over the distal end and point of needle 201. Shroud 208 can be a measured section of semi-rigid tubing and can be sized such that shroud 208 can be inserted into a shroud receptacle or pocket at the face of needle shield 240, where shroud 208 can be held in place by a friction fit. When a venipuncture is to occur, shroud 208 can be removed to expose needle 201.

The present disclosure also contemplates needle assemblies that do not include an extendable safety shield and instead have a retractable needle that can be retracted upon activation of an actuator (e.g., a button or switch). Such embodiments can benefit from the disclosed needle protectors that can prevent accidental retraction of a needle before, during, or after venipuncture of a patient.

Needle shield 240 can be deployed by operation of a shield deployment mechanism, described in further detail below, which, driven by spring 250 as it releases and expands, deploys, pushes, or forces needle shield 240 from the packaged mode to the safety mode, in which needle shield 240 completely covers the distal end of needle 201 and shields it from causing inadvertent needle sticks. Needle assembly 200 further includes a shield locking mechanism to lock the shield in the safety mode of the needle assembly 200 (e.g., where the needle shield 240 covers the needle 201 and is locked in that position), as described further below.

The hub 230 is fixed at least partially within housing 202 at a proximal end 207 of housing 202 opposite needle 201, and to which outer tubing 203 and inner tubing 205 are connected. Outer tubing 203 can be connected with a fluid collection device, a blood sample optimization device, or any other device or receptacle.

Hub 230 can be secured to housing 202 by hub flange 232 or catch or post that extends outward from a main body of hub 230, and which occupies or otherwise connects to or within housing aperture 204 formed in housing 202. Hub flange 232 can have an angled forward face and a slightly angled rearward face, as shown in FIGS. 2A and 2B, so as to be easily inserted into the proximal end of housing 202 during manufacturing and secured in place in housing aperture 204 upon insertion of hub 230 into housing 202. The angled rearward face prevents the flexible wall of housing 202 from bending and allowing hub 230 to be pushed out of or otherwise released from housing 202.

Hub 230 connects with outer tubing 203 at an outer or proximal face of the hub 230 at a proximal end 207 of housing 202. Outer tubing 203 can be positioned inside a cylindrical cavity of the hub 230, as shown in FIGS. 2A and 2B, and/or secured by an adhesive 236. An inner face of hub 230 can be connected directly with needle 201 or via an inner tubing 205 on a distal side (where needle 201 is). Inner tubing 205 preferably has a similar or smaller internal diameter as outer tubing 203, which can reduce the risk of hemolysis, for example. The inner tubing 205 is in turn connected and fluidically sealed to needle 201 within housing 202, such that needle 201 extends out of a distal end 209 of housing 202.

The hub 230 further includes a forward abutment region 234, or distal face, on the inner face of the hub 230, and on which a proximal, or first, end of a spring 250 can abut, be placed or connected, and leveraged against. Spring 250 can be a coiled spring that is coiled, placed or fits around the inner tubing 205 and can be positioned over at least some of the needle 201. In some implementations, the forward abutment region 234 of hub 230 can be formed as a ring around inner tubing 205, to provide a maximal surface area on which the spring can abut. The forward abutment region 234 of hub 230 therefore provides an immovable surface against which spring 250 can release, push, react, or the like.

In the packaged mode, needle shield 240 can be secured within housing 202 by a securement mechanism to expose the needle 201, which extends from both the needle shield 240 and a distal end 209 of housing 202. The shield securement mechanism can include a lock-in latch 210 as part of housing 202 that extends into an inner chamber of housing 202, and which mates with a securement latch 244 as part of needle shield 240 proximate a distal end 246 of a flexible and depressible shield securement arm 245, which can also be formed as a cut-out portion of the material the needle shield 240, as described in further detail below.

Needle shield 240 can include an abutment member 248, such as inwardly-directed protrusion, near or at a distal end of needle shield 240, on which a distal end of spring 250 can abut, be placed or connected, and leveraged, pushed, or reacted against. Abutment member 248 can be formed by an inward-projecting ring or ridge and provide a rearward face to abut the distal or second end of spring 250. Abutment member 248 can also fix or stabilize needle 201 and/or any associated tubing in a longitudinal position relative to housing 202. Accordingly, spring 250 can exert a bias or force against the inner face of abutment member 248.

In the packaged mode, lock-in latch 210 of housing 202 can be mated with the securement latch 244 of the needle shield 240, and spring 250 is compressed between the forward abutment region 234 of the hub and the abutment member 248 of the shield. Securement latch 244 can be latched, pressed, or forced against lock-in latch 210 in the direction of the force of the compressed spring 250 (e.g., from the hub toward a distal end 209 of the housing 202 and the needle 201).

The shield deployment mechanism is implemented by a cooperation between housing 202, the needle shield 240 and the spring 250. In some implementations, housing 202 includes button 222 for activating a deployment of the needle shield 240. Button 222 can be a raised portion of a flexible and depressible activation arm 224. The activation arm 224 can be formed as a portion of the material that forms the housing 202, as described in further detail below. In some implementations, the housing 202 includes a cut-out space between the button 222 and the lock-in latch 210 to allow space for the securement latch 244 on the securement arm 245 of the needle shield 240 to extend upward and latch against the lock-in latch 210 of the housing 202.

In some implementations, at least part of button 222 is positioned above the distal end 246 of the flexible securement arm 245. The distal end 246 forms a platform against which button 222, when depressed by a technician, pushes distal end 246 and securement arm 245 down to release the securement latch 244 from abutting against the lock-in latch 210. The distal end 246 can include a downward-sloping shape from the securement latch 244 toward the terminus of the distal end 246, so as to facilitate a smooth and effective disengagement of securement latch 244 from lock-in latch 210.

Upon disengagement of securement latch 244 from lock-in latch 210, needle shield 240 will slide distally away from hub 230 and out of distal end 209 of housing 202, by force of the compressed spring 250 which begins to expand to its original length. Housing 202 can have one or more longitudinal indented grooves 252, channels or tracks, which can extend at least partially along the internal walls of the housing 202, for guiding the sliding and deploying needle shield 240, which can have corresponding tabs or guides that extend out from an outer surface of needle shield 240. Alternatively, needle shield 240 can have one or more grooves, channels, or tracks, while an inner surface of housing 202 can have corresponding tabs or guides. The tracks and guides allow for proper placement and orientation of needle shield 240 relative to housing 202. In embodiments where both an inner cross section of housing 202 and an outer cross section of needle shield 240 are circular, this can prevent rotation of needle shield 240 relative to housing 202. In other embodiments, both an inner cross section of housing 202 and an outer cross section of needle shield 240 can have one or more non-circular shapes, such as an angle or compressed curve, which can also prevent rotation of needle shield 240 relative to housing 202 as needle shield 240 is deployed. These tracks, guides, and tabs also cooperate to stop or limit the forward movement of needle shield 240 from housing 202, so that needle shield 240 does not become disconnected or eject from housing 202, as will be described in further detail below.

As needle shield 240 slides forward relative to housing 202 and away from hub 230, a bottom surface 221 of needle shield 240 can be configured, by virtue of a ramped front end 223 of the bottom surface 221, to slide over a lock-out latch 206 of housing 202. Lock-out latch 206 can have a forward locking face 239 that is configured to eventually mate against a rear face 242 of needle shield 240, such as at bottom surface 221 of needle shield 240, or against tabs that extend out from the needle shield 240. In the packaged mode, lock-out latch 206 of housing 202 protrudes up from an inner surface of the housing to occupy a gap 220 in needle shield 240 proximate the ramped front end 223 of bottom surface 221 of needle shield 240.

In some implementations, the outer surface of needle shield 240 and the inner surface of the housing, at least at the top and bottom of the shield, are configured to be spaced apart to allow easy deployment of needle shield 240 from housing 202, as well as allow bottom surface 221 of needle shield 240 to slide over lock-out latch 206, which can be at the end of a cut-out arm formed in the bottom of housing 202 so as to bend lock-out latch 206 flexibly out of the way of the bottom surface 221 of needle shield 240 as it slides over the lock-out latch 206. This spacing can also allow securement latch 244 of needle shield 240 to slide out of the top inner surface of housing 202. Needle shield 240 further includes a channel 226 or groove, opening, or the like, in a surface of needle shield 240, such as the top surface, to allow the needle shield 240 to deploy and slide past the depressed button 222, which can extend at least partially into channel 226.

Needle shield 240 can slide forward to deploy over and cover the needle 201 where the forward movement of needle shield 240 can be stopped by a cooperation of the tabs and guides, whether on the inner surface of the housing 202 or outer surface of the needle shield 240, so that the needle shield 240 does not become disconnected or eject from the housing 202.

Once needle shield 240 has fully deployed in an extended position (e.g., to the maximum extent allowed by the one or more tracks and one or more tabs) the rear face 242 of the bottom surface 221 of needle shield 240 moves beyond and forward of or distal to the lock-out latch 206, which returns toward the inner portion of the housing to engage the forward locking face 239 of the lock-out latch 206. Accordingly, needle shield 240 cannot be pushed back into or otherwise retracted back inside housing 202, and needle shield 240 will be locked to cover needle 201. In some implementations, needle assembly 200 is configured such that the spring abutment member 248 of needle shield 240, against which the distal end of spring 250 is attached, extends beyond the distal end or tip of needle 201, whether or not further coverage by needle shield 240 extends further beyond the distal end or tip of needle 201.

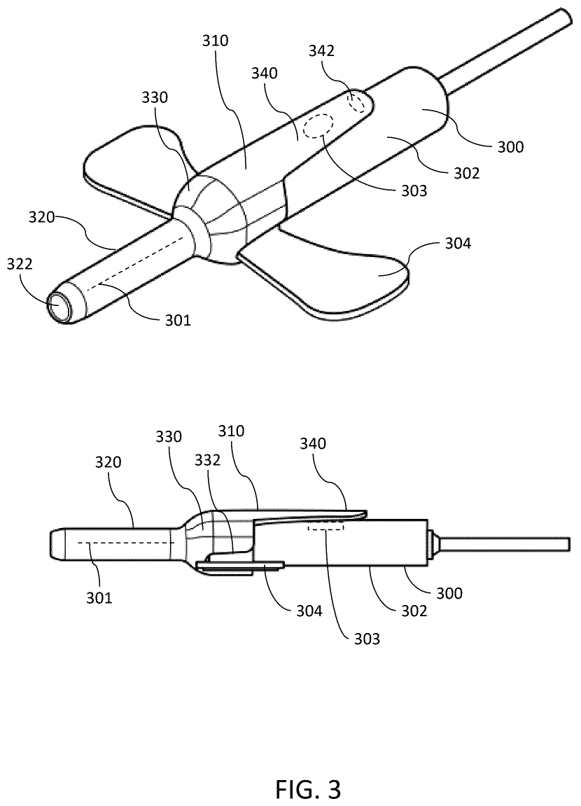

FIG. 3 is a diagram depicting a simplified perspective and side view of a needle protector. Needle protector 310 can be used with a needle assembly 300, for example, those shown in FIGS. 1, 2A, and 2B. FIG. 3 shows needle assembly including a needle 301, a housing 302, from which needle 301 extends, and an actuator 303 for activating a safety mechanism following use of needle 301. Examples of actuators can include buttons, tabs, switches, knobs, etc.

As used herein, the term “safety mechanism” refers to a broad class of structural features that facilitate reducing the likelihood of inadvertent needle sticks. For example, a safety mechanism can be a mechanism to extend a safety shield (e.g., the previously mentioned shield deployment mechanism), a mechanism to retract the needle into the housing, etc. While these two examples are described in detail herein, a person of skill understands that in various embodiments there can be a variety of safety mechanisms. Accordingly, an actuator can be configured to activate or control any sort of safety mechanism for a needle assembly. Also, an actuator can be configured to be coupled with mechanisms of a needle assembly that are not safety mechanisms. For example, in some embodiments, an actuator can trigger disassembly, release, or locking of one or more components, activation of a light or other electronic feature, etc. The disclosed needle protectors can thereby facilitate avoidance of inadvertent actuations of these features as well.

The exemplary embodiment of needle protector 310 shown in FIG. 3 can include a sleeve 320 configured with a lumen 322 sized to accept needle 301 and a length that extends at least partially over needle 301. Needle protector 310 can also include a cuff 330 connected to sleeve 320 having a cavity shaped to receive a portion of the housing. Needle protector 310 can further include an actuator cover 340 extending from cuff 330, opposite sleeve 320, and configured to cover actuator 303 when needle protector 310 is in place over needle 301. The depicted actuator 303 is a simplified example to illustrate how actuator cover 340 can cover actuator 303 to prevent unintentional actuation.

In various embodiments, actuator cover 340 can be configured to prevent actuation of actuator 303 by incorporating various features either alone or in combination. For example, the actuator cover 340 can have rigidity for preventing the actuation. The rigidity of actuator cover 340 can be obtained by a combination of the material of actuator cover 340 (e.g., plastic, metal, etc.), the dimensions of actuator cover 340 (e.g., thickness, width, length), or rigidity enhancing (or reducing) features such as support ribs, cutouts, etc. The rigidity can result in actuator cover 340 resisting accidental actuation (e.g., a rigidity such that actuation can only result with increased force due to intentional effort). In other embodiments, the rigidity can be increased such that actuation is effectively impossible without removing needle protector 310 (e.g., actuator cover 340 made of very rigid plastic or metal). In some embodiments, preventing actuation can be further facilitated by actuator cover 340 including a protrusion 342 located on an inside surface of actuator cover 340 and extending towards the needle assembly 300 for maintaining a gap between actuator cover 340 and actuator 303.

Needle protector 310 can be removably secured to needle assembly 300. For example, cuff 330 can be configured to have a friction fit with housing 302 of needle assembly 300. In this way, cuff 330 can be secured against housing 302 in one or more locations until needle protector 310 is pulled off needle assembly 300. In some embodiments, needle assembly 300 can include wings 304 that have been secured to the housing (e.g., via collar 110). Cuff 330 can then include a slot 332 shaped to accept wings 304 of needle assembly 300. Slot 332 can be configured to have a friction fit with wings 304 such that the sides of the slot press against upper and/or lower surfaces of wings 304. In another embodiment, where collar 110 is larger than housing 302, cuff 330 can be configured to have a friction fit with collar 110.

Sleeve 320 can have a length sufficient to cover needle 301. Such a length can cause an opening to lumen 322 to be at the tip of needle 301. In some embodiments, the length can extend further (e.g., 1 mm, 2 mm, 5 mm, etc.) such that the tip of needle 301 is recessed within sleeve 320. The radial dimension of the inner surface of lumen 322 can vary to be, for example, a close fit to a needle radius, or have more of a clearance fit or even larger (e.g., an extra 0.5 mm, 1 mm, 2 mm, etc.). In some embodiments, a distal end of sleeve 320 can have a tapered or rounded section as depicted in FIG. 3.

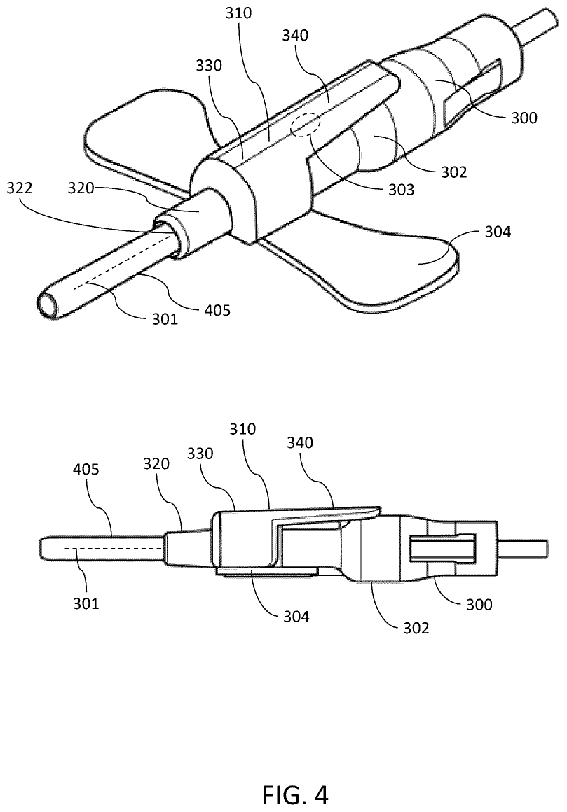

FIG. 4 is a diagram depicting a simplified perspective and side view of a needle protector over a needle shroud. Some needle assemblies may include a needle shroud 405 that covers needle 301. Accordingly, some embodiments of needle protector 310 can have lumen 322 of sleeve 320 sized to accept both needle 301 and needle shroud 405. As shown in FIG. 4, in some embodiments, the length of sleeve 320 extends only partially over needle 301 and needle shroud 405, leaving some of needle 301 and needle shroud 405 exposed. In other embodiments, the length of sleeve 320 can extend completely over needle 301 and needle shroud 405, similar to that depicted in FIG. 3. In some embodiments, lumen 322 can be further sized to form a press fit around needle shroud 405 to cause needle shroud 405 to be removed from needle 301 along with needle protector 310. In some embodiments, the inner surface of lumen 322 can be shaped as a frustrum of a cone where the narrow (distal) end is a friction fit. In other embodiments, the inner surface of lumen 322 can be a cylinder where most or all of the cylindrical surface engages needle shroud 405.

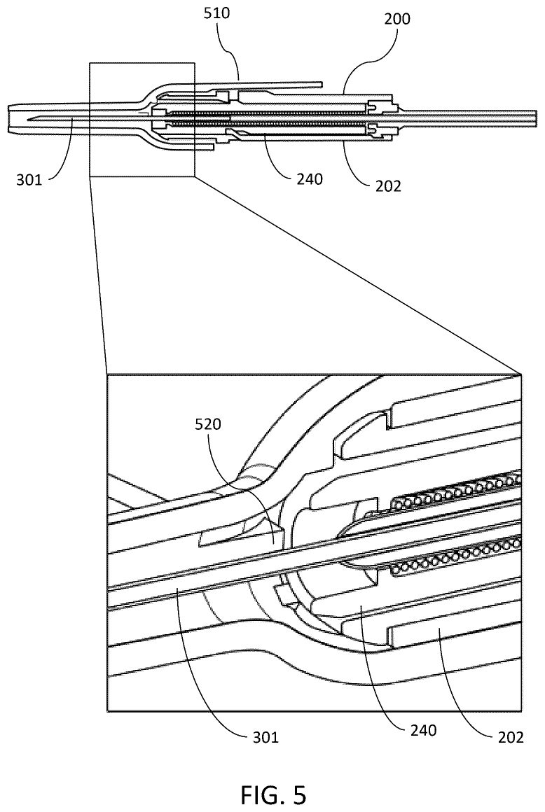

FIG. 5 is a diagram depicting a simplified side sectional view of a needle protector configured to reset an extended needle shield. Some needle assemblies can include extendable and retractable needle shields that can be deployed to cover a needle and prevent inadvertent needle sticks. One example of such a needle assembly is depicted in FIGS. 2A/B. A simplified reproduction of this embodiment is depicted in FIG. 5 showing needle assembly 200 and needle shield 240 in a retracted state. To facilitate resetting/retracting of such a needle assembly, some embodiments of a needle protector 510 can include a rib 520 extending inwardly and configured to engage an extended needle shield 240 in order to reset an extended needle shield 240. Rib 520 can abut a distal face of needle shield 240 and push needle shield 240 back into housing 202 when needle protector 510 is pushed in the proximal direction (towards the rear of needle assembly 200) by a user. Such a resetting operation can occur when, for example needle shield 240 is actuated but not allowed to fully extend such that a lock-out feature (e.g., lock-out latch 206) engages. Another example is using needle protector 510 to reset needle assemblies that do not have lock-out features.

In some embodiments, the lock-out features can be mechanically reversed or reset by a variation of needle protector 510. For example, a lock-out latch 206, when engaged, may be disposed inwardly such that a portion of needle shield 240 abuts the lock-out latch and cannot be pushed or retracted back into housing 202 and re-expose needle 301. In this variation, needle protector 510 can be configured to mechanically engage lock-out latch 206 to pull it outward as needle protector 510 is slid onto needle assembly 200. With lock-out latch 206 disengaged, needle shield 240 can be pushed back into its original position by needle protector 510.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, computer programs and/or articles depending on the desired configuration. Any methods or the logic flows depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

What is claimed is:

1. A needle protector for use with a needle assembly that includes a needle, a housing from which the needle extends, and an actuator for activating a safety mechanism following use of the needle, the needle protector comprising:
a sleeve configured with a lumen sized to accept the needle and a length that extends at least partially over the needle;
a cuff connected to the sleeve having a cavity shaped to receive a portion of the housing; and
an actuator cover extending from the cuff, opposite the sleeve, and configured to cover the actuator when the needle protector is in place over the needle.

2. The needle protector of claim 1, wherein the actuator cover is configured to prevent actuation of the actuator.

3. The needle protector of claim 2, wherein the actuator cover has a rigidity for preventing the actuation.

4. The needle protector of claim 2, wherein the actuator cover includes a protrusion extending towards the needle assembly for maintaining a gap between the actuator cover and the actuator.

5. The needle protector of claim 1, wherein the cuff is configured to have a friction fit with the housing of the needle assembly.

6. The needle protector of claim 1, wherein the cuff includes a slot shaped to accept wings of the needle assembly.

7. The needle protector of claim 6, wherein the slot is configured to have a friction fit with the wings.

8. The needle protector of claim 1, wherein the sleeve has a length sufficient to cover the needle.

9. The needle protector of claim 1, wherein the lumen of the sleeve is sized to accept both the needle and a needle shroud.

10. The needle protector of claim 9, wherein the length of the sleeve extends only partially over the needle and the needle shroud, leaving some of the needle and the needle shroud exposed.

11. The needle protector of claim 9, wherein the length of the sleeve extends completely over the needle and the needle shroud.

12. The needle protector of claim 9, wherein the lumen is further sized to form a press fit around the needle shroud to cause the needle shroud to be removed from the needle along with the needle protector.

13. The needle protector of claim 1, further comprising a rib extending inwardly and configured to engage an extended needle shield in order to reset the extended needle shield.
